(19) United States
(12) Patent Application Publication
(10) Pub. No.: US 2015/0140101 A1
(43) Pub. Date: May 21, 2015

(54) SOLID ORAL COMPOSITIONS OF SILODOSIN

(71) Applicant: HETERO RESEARCH FOUNDATION, Andhra Pradesh (IN)

(72) Inventors: Bandi Parthasaradhi Reddy, Hyderabad (IN); Podili Khadgapathi, Hyderabad (IN); Goli Kamalakar Reddy, Hyderabad (IN)

(21) Appl. No.: 14/412,110

(22) PCT Filed: Jun. 27, 2013
(86) PCT No.: PCT/IN13/00395

§ 371 Date: Dec. 30, 2014

(30) Foreign Application Priority Data
Jul. 2, 2012 (IN) 2631/CHE/2012

(51) Int. Cl. A61K 31/404; A61K 9/14; A61K 47/38; A61K 47/36; A61K 9/48; A61K 47/12; A61K 47/26

(52) U.S. Cl. CPC A61K 31/404; A61K 47/12; A61K 9/14; A61K 47/26; A61K 47/36; A61K 9/4833; A61K 9/4858; A61K 9/4866; A61K 47/38
USPC 424/489; 514/415

(57) ABSTRACT

The present disclosure relates to solid oral compositions of silodosin or its pharmaceutically acceptable salt thereof. More particularly capsule compositions of silodosin with one or more pharmaceutically acceptable excipients and process for their preparation.

### PRIORITY

This patent application claims priority to Indian patent application number 2631/CHE/2012, filed on Jul. 2, 2012, the contents of which are incorporated by reference herein in their entirety.

### FIELD OF THE INVENTION

The present invention encompasses solid oral compositions of silodosin or its pharmaceutically acceptable salt thereof. More particularly capsule compositions comprising silodosin and one or more pharmaceutically acceptable excipients.

### BACKGROUND OF THE INVENTION

Chemically silodosin is 1-(3-Hydroxypropyl)-5-[(2R)-2-({2-[2-(2,2,2trifluoroethoxy)phenoxy]ethyl}amino) propyl]-2,3-dihydro-1H-indole-7-carboxamide. Its empirical formula is C₂₅H₃₂F₃N₃O₄, corresponding to a molecular weight of 495.53 having the following structural formula:

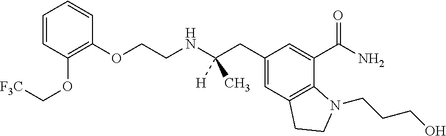 [SMILES: C[C@H](Cc1cc2c(c(C(N)=O)c1)N(CCCO)CC2)NCCOc1ccccc1OCC(F)(F)F]

Silodosin is marketed under the trade name RAPAFLO® in United States by Watson Labs in the form of 4mg and 8 mg capsules for the treatment of the signs and symptoms of benign prostatic hyperplasia.

U.S. Pat. No. 5,387,603 assigned to Kissei pharmaceutical disclose silodosin.

U.S. Pat. No. 5,403,847, U.S. Pat No. 5,780,485 and U.S. Pat. No. 6,015,819 assigned to Synaptic pharmaceutical claims silodosin for treating benign prostatic hyperplasia.

WO2012010669 and WO201200926 assigned to Ratiopharm claims composition comprising a mixture of silodosin with acrylate or methacrylate copolymer; and an inclusion compound of silodosin with cyclodextrin respectively.

US20120064154 assigned to Kissei pharmaceutical claims capsule composition of silodosin with a lubricant selected from magnesium stearate, calcium stearate or talc; and sodium lauryl sulfate as surfactant.

Use of a lubricant in composition of silodosin is inevitable because silodosin has potent adhesive property. Accordingly, prior art compositions contain lubricants such as magnesium stearate, calcium stearate and the like. On the, contrary, addition of such lubricants cause the problem of delaying the dissolution time. To overcome such dissolution problem, surfactants like sodium lauryl sulphate was used to improve the dissolution properties. However, the effect of surfactants over the intestinal membrane is more complex. It has been shown that most surfactants interact with the absorbing membranes. Permeability enhancement and local damage are closely related sequelae of the interaction of surfactants with the intestinal wall. Further, the use of surfactants may facilitate penetration or absorption of endotoxins or pathogenic compounds in to the systemic circulation, which in turn may result in adverse effects.

Thus, there is a need to have compositions of silodosin with better dissolution properties without use of surfactant. Accordingly, inventors of the present invention have eagerly investigated novel compositions of silodosin using lubricants, which have no effect on dissolution rate.

### SUMMARY OF THE INVENTION

The present invention provides solid oral compositions comprising silodosin or a pharmaceutically acceptable salt thereof and process for their preparation.

One embodiment of the present invention includes solid oral composition comprising silodosin, a lubricant selected from sodium stearyl fumarate, polyethylene glycol, and polyvinyl alcohol or combinations thereof and one or more pharmaceutically acceptable excipients.

Another embodiment of the present invention includes solid oral composition comprising silodosin having a particle size distribution d₉₀ of 1 μ to 25 μ, more preferably from 10 μ to 20 μ.

In embodiments, capsule composition comprises silodosin as an active agent, sorbitol as diluent, sodium stearyl fumarate as lubricant and one or more excipients selected from pregelatinized starch and croscarmellose sodium.

In another embodiment, the present invention also provides a capsule composition comprising silodosin as an active agent, xylitol as diluent, sodium stearyl fumarate as lubricant and one or more excipients selected from pregelatinized starch and croscarmellose sodium.

In an embodiment of the present invention provides method of making a silodosin composition involves: (i) sifting and blending silodosin with one-or more pharmaceutically acceptable excipients to form a uniform blend, (ii) lubricating the blend of step (i) with sodium stearyl fumarate and (iii) filling the lubricated blend of step (ii) into capsules.

In another embodiment of the present invention provides use of silodosin composition for treating the signs and symptoms of benign prostatic hyperplasia.

### DETAILED DESCRIPTION OF THE INVENTION

The present invention relates to pharmaceutical compositions comprising silodosin or a pharmaceutically acceptable salt thereof and process for their preparation.

The term “silodosin” as used herein according to the present invention includes silodosin in the form of free base, a pharmaceutically acceptable salt thereof, amorphous silodosin, crystalline silodosin, any isomer, derivative, hydrate, solvate or prodrug or a combination thereof.

The term “active ingredient” herein refers to a pharmaceutically active molecule as well as its pharmaceutically acceptable and therapeutically active salts, esters, amides, prodrugs, metabolites, enantiomers, polymorphs, analogs, etc. that induce a desired pharmacological or physiological effect. Terms like “active”, “active agent”, “active substance” may be used synonymously for “active ingredient”.

The term “effective amount” or “therapeutically effective amount” used interchangeably, is defined to mean the amount or quantity of the active drug (e.g. silodosin), which is sufficient to elicit an appreciable biological response when administered to the patient. It will be appreciated that the precise therapeutic dose will depend on the age and condition of the patient, nature of the condition to be treated and will be at the ultimate discretion of the attendant physician.

The term “excipient” means a pharmacologically inactive component such as a diluent, disintegrant, carrier, and the like, of a pharmaceutical product. The excipients that are useful in preparing a pharmaceutical composition are generally safe, non-toxic and are acceptable for veterinary as well as human pharmaceutical use. Reference to an excipient includes both one excipient and more than one excipient.

The term “composition” or “solid oral composition” or “dosage form” as used herein synonymously include solid dosage forms such as capsules, powder, particles, granules, pellets, tablets, mini-tablets and the like meant for oral administration.

Generally the pharmaceutical compositions of the present invention prepared in unit dosage forms are meant for immediate release.

As used in this specification and the appended claims, the singular forms “a”, “an”, and “the” include plural references unless the context clearly dictates otherwise. Thus for example, a reference to “a method” or “a process” includes one or more methods, one or more processes and/or steps of the type described herein and/or which will become apparent to those persons skilled in the art upon reading this disclosure and so forth.

The present invention relates to a solid oral compositions comprising silodosin with one or more pharmaceutically acceptable excipients and process for their preparation.

In one aspect, solid oral composition comprises silodosin, a lubricant selected from sodium stearyl fumarate, polyethylene glycol, and polyvinyl alcohol or combinations thereof and one or more pharmaceutically acceptable excipients.

From the available prior arts it has been known that compositions of silodosin containing lubricants such as magnesium stearate, calcium stearate, and talc have an effect on the dissolution rate with mixing time. Accordingly inventors of the present invention have formulated novel compositions of silodosin using lubricants, which have no effect on dissolution rate with mixing time.

Lubricants according to the present invention include but are not limited to sodium stearyl fumarate, polyethylene glycol, and polyvinyl alcohol or combinations thereof, preferably sodium stearyl fumarate.

Preferred concentration of sodium stearyl fumarate used according to the present invention is from 0.1 to 5 wt % based on total weight of the composition.

Particularly, the present invention relates to surfactant free compositions of silodosin with desired dissolution profile, prepared by using lubricants which have no effect on dissolution rate.

In an aspect, a capsule composition comprises silodosin as an active ingredient, sorbitol as diluent, sodium stearyl fumarate as lubricant and one or more excipients selected from pregelatinized starch and croscarmellose sodium or a combination thereof.

In another aspect, a capsule composition comprises silodosin as an active agent, xylitol as diluent, sodium stearyl fumarate as lubricant and one or more excipients selected from pregelatinized starch and croscarmellose sodium.

Accordingly, the present invention relates to a capsule composition comprising, based on total weight of the composition, i) 1 to 8 wt % of silodosin, ii) 60 to 90 wt % sorbitol or xylitol, iii) 0.1 to 5 wt % sodium stearyl fumarate and iv) one or more excipients selected from pregelatinized starch and croscarmellose sodium or a combination thereof.

Particle size plays an important role in establishing solubility and dissolution of silodosin. As the particle size is reduced, the surface area of the individual particles of silodosin increase, which provides good in vitro end release and invivo bioavailability.

Silodosin having a particle size d₉₀ of 1 μ-25 μ, more preferably from 10 μ-20 μ is used in preparing pharmaceutical composition.

Desired particle size of silodosin was obtained by any suitable micronization technique known in the art such as dry milling, wet milling, air jet milling, sieving etc.

The solid oral compositions include any number of excipients, including, but not limited to, diluents or fillers, binders, disintegrants, glidants and mixtures thereof.

Diluents/Fillers: Various useful fillers or diluents include but are not limited to sorbitol, xylitol, pregelatinized starch, maize starch, potato starch, rice starch, wheat starch, powdered celluloses, dibasic calcium phosphate, calcium phosphate, calcium carbonate, magnesium carbonate, microcrystalline cellulose and the like and mixtures thereof; more preferably selected from sorbitol and xylitol.

Binders: Various useful binders include but are not limited to pregelatinized starch, hydroxypropyl cellulose, hydroxypropyl methylcellulose, polyvinyl pyrrolidone, powdered acacia, gelatin, guar gum, carbomers.

Disintegrants: Various useful disintegrants include but are not limited to pregelatinized starch, croscarmellose sodium, crospovidone, sodium starch glycolate and the like.

Glidants: One or more glidants, which improve the flow of a powder blend can be used. Useful glidants include but are not limited to, colloidal silicon dioxide, magnesium silicate, magnesium trisilicate, talc, and other forms of silicon dioxide, such as aggregated silicates and hydrated silica.

Solid oral composition according to the present invention is free of sodium lauryl sulphate, magnesium stearate, calcium stearate, talc and the like or a combination thereof

In embodiments of the present invention, equipment suitable for processing the pharmaceutical formulations include one or more of mechanical sifters, granulators, blenders, compression machines, fluid bed processors, etc.

In embodiments of the present invention, the pharmaceutical formulations may be processed by simple mixing and filling into capsules or alternatively granules prepared by wet granulation were filled into capsules.

In one aspect, the present invention includes method of making a silodosin composition involves (i) sifting and blending silodosin with one or more pharmaceutically acceptable excipients to form a uniform blend, (ii) lubricating the blend of step (i) with sodium stearyl fumarate and (iii) filling the lubricated blend of step (ii) into capsules or compressing into tablets.

Method of making silodosin composition by wet granulation involves (i) sifting and blending silodosin with one or more pharmaceutically acceptable excipients to form a uniform blend, (ii) granulating the blend of step (i) using a solvent or binder solution, followed by drying and sizing to get desired granules, (iii) lubricating the granules of step (ii) with a lubricant and (iv) finally filling the lubricated granules of step (iii) into capsules or compressing into tablets.

Dosage forms prepared by the above process can be subjected to in vitro dissolution test to determine the extent and rate at which the active substance is released from the dosage forms and the content of the active substance can be determined in solutions by using techniques such as high performance liquid chromatography.

Since silodosin and its compositions are light sensitive, they are generally stored in a light-resistant packaging. Accordingly suitable packaging materials include amber colour high-density polyethylene (HDPE) containers, white opaque high-density polyethylene (HDPE) containers, aluminum/aluminum foil blisters and white opaque polyvinyl chloride/polyvinylidene chloride (PVC/PVdC) blisters.

Solid oral compositions of the present invention comprising therapeutically effective amount of silodosin is useful in treating the signs and symptoms of benign prostatic hyperplasia.

### BRIEF DESCRIPTION OF THE DRAWINGS

FIG. 1 is showing the effect of mixing time of lubricant on dissolution rate.

FIG. 2 is showing relationship between particle size of silodosin and dissolution rate.

Certain specific aspects and embodiments of this invention are described in further detail by the examples below, which are provided only for purposes of illustration and are not intended to limit the scope of the invention in any manner.

### Example 1

Study of relationship between mixing time of lubricant and dissolution rate.

The correlation between mixing time of lubricant for 3, 5 and 7 minutes and dissolution rate was investigated using capsules containing sodium stearyl fumarate as lubricant. Each capsule was prepared according to the formulation shown in Table 1, and their dissolution results were presented in Table 2.

TABLE 1

| | Example 1 |
|---|---|
| Ingredient | mg/unit |
| Silodosin | 8.00 |
| Sorbitol | 296.00 |
| Pregelatinized starch | 40.00 |
| Sodium stearyl fumarate | 6.00 |
| Fill weight | 350.00 |

### Brief Manufacturing Process:

1. Silodosin, sorbitol and pregelatinized starch were sifted through mesh #40 and blended for around 15 minutes,
2. sodium stearyl fumarate was sifted through mesh #40,
3. blend of step 1 was lubricated with sodium stearyl fumarate of step 2 and
4. lubricated blend of step 3 was filled into capsules.

### Dissolution Study:

### Dissolution Medium: 0.1N HCL

### Volume: 900 ml

### Apparatus: USP II (Sinkers)

### Speed: 50 RPM

TABLE 2

| | Example 1/ | Example 1/ | Example 1/ |
|---|---|---|---|
| Time in minutes | 3 min mixing | 5 min mixing | 7 min mixing |
| 5 | 80 | 83 | 84 |
| 10 | 94 | 95 | 96 |
| 15 | 97 | 96 | 96 |
| 20 | 99 | 96 | 97 |
| 30 | 99 | 97 | 99 |
| 45 | 99 | 97 | 99 |

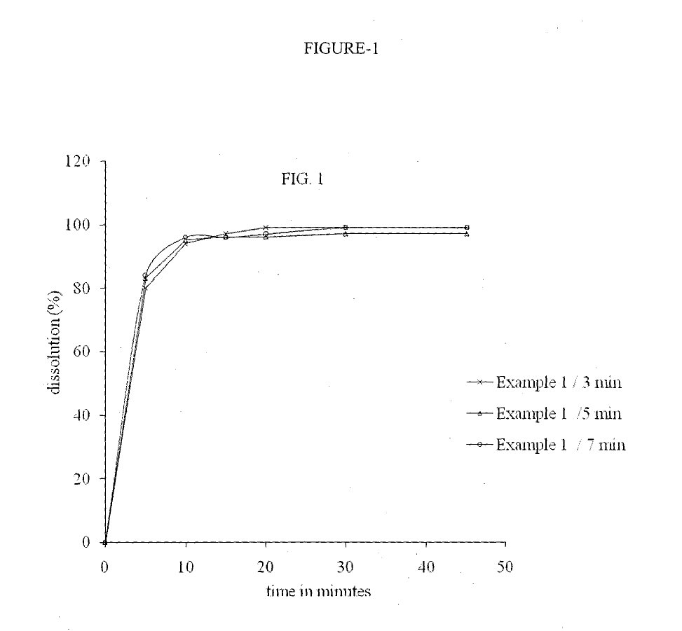

As can be seen from Table 2 and/or FIG. 1, Example 1 containing sodium stearyl fumarate as lubricant showed excellent dissolution properties with no effect of mixing time.

### Example 2

Study of relationship between particle size of silodosin and dissolution rate.

The correlation between particle size of silodosin and dissolution rate was investigated by using capsules containing silodosin with various particle size diameter. Each capsule was prepared according to the formulation shown in Table 3, and their dissolution results were presented in Table 4.

TABLE 3

| | Formulation ‘A’ | Formulation ‘B’ |
|---|---|---|
| | mg/unit | mg/unit |
| | Silodosin having d₉₀ = | Silodosin having d₉₀ = |
| Ingredient | 12μ | 40μ |
| Silodosin | 8.00 | 8.00 |
| Sorbitol | 296.00 | 296.00 |
| Pregelatinized starch | 40.00 | 40.00 |
| Sodium stearyl fumarate | 6.00 | 6.00 |
| Fill weight | 350.00 | 350.00 |

Manufacturing process: Same as Example-1.
Dissolution study:

### Dissolution Medium Water

### Volume: 900 ml

### Apparatus: USP II (Sinkers)

### Speed: 50 RPM

TABLE 4

| | Formulation ‘A’ | Formulation ‘B’ |
|---|---|---|
| Time | mg/unit | mg/unit |
| in minutes | Silodosin having d₉₀ = 12μ | Silodosin having d₉₀ = 40μ |
| 5 | 83 | 48 |
| 10 | 95 | 72 |
| 15 | 96 | 77 |
| 20 | 96 | 78 |
| 30 | 97 | 80 |
| 45 | 97 | 83 |

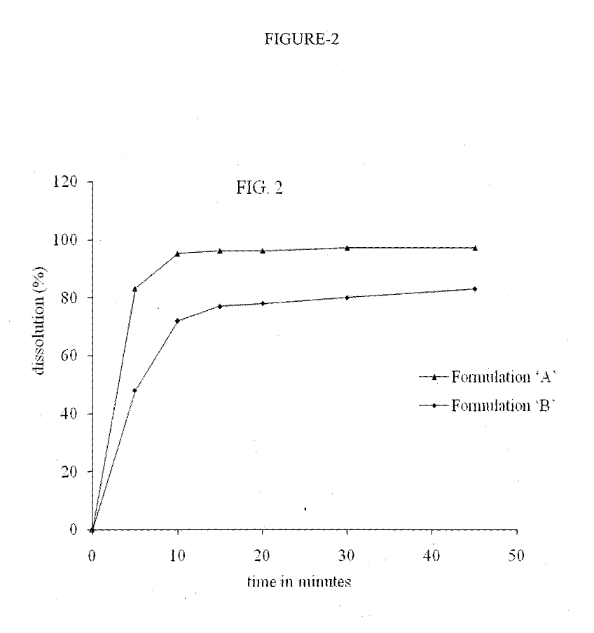

to As can be seen from Table 4 and/or FIG. 2, Formulation ‘A’ containing silodosin d₉₀ particle size of 12 μ showed excellent dissolution properties with better end release as compared to Formulation ‘B’ containing silodosin d₉₀ particle size of more than 25 Based on the results presented in Table 4, the dissolution rate of silodosin has increased, as the particle size is reduced.

### Example 3-4

Solid oral compositions of silodosin:

| | Example 3 | Example 4 |
|---|---|---|
| Ingredient | mg/unit | mg/unit |
| Silodosin | 4.00 | 8.00 |
| Sorbitol | 141.00 | — |
| Xylitol | — | 282.00 |
| Pregelatinized starch | 20.00 | 40.00 |
| Croscarmellose sodium | 7.00 | 14.00 |
| Sodium stearyl fumarate | 3.00 | 6.00 |
| Fill weight | 175.00 | 350.00 |

### Manufacturing Process:

1. Silodosin, sorbitol/xylitol, pregelatinized starch and croscarmellose sodium were sifted through mesh # 40 and blended for around 15 minutes,
2. sodium stearyl fumarate was sifted through mesh #40,
3. blend of step 1 was lubricated with sodium stearyl fumarate of step 2 and
4. lubricated blend of step 3 was filled into capsules or compressed into tablets.

### Example 5

Solid oral compositions of silodosin prepared by wet granulation method:

| Ingredient | mg/unit |
|---|---|
| Silodosin | 8.00 |
| Sorbitol | 296.00 |
| Pregelatinized starch | 40.00 |
| Purified water | q.s |
| Sodium stearyl fumarate | 6.00 |
| Fill weight | 350.00 |

### Manufacturing Process:

1. Silodosin, sorbitol and pregelatinized starch were sifted through mesh #40 and blended,
2. blend of step 1 was granulated using purified water,
3. the wet mass of step 2 was dried and milled to get the desired granules,
4. sodium stearyl fumarate was sifted through mesh #40,
5. granules of step 3 were lubricated with sodium stearyl fumarate of step 4 and
6. lubricated granules of step 5 were filled into capsules or compressed into tablets.

1. A solid oral composition comprising:
i) silodosin,
ii) sodium stearyl fumarate as a lubricant, and
iii) one or more pharmaceutically acceptable excipients.

2. The solid oral composition according to claim 1, in the form of a capsule or a tablet.

3. The solid oral composition according to claim 1, wherein said sodium stearyl fumarate is present in an amount of 0.1 to 5 wt % based on total weight of the composition.

4. The solid oral composition according to claim 1, wherein said pharmaceutically acceptable excipient is selected from a diluent, a binder, a disintegrant and a glidant, and or a combinations thereof.

5. The solid oral composition according to claim 1, wherein saidpharmaceutically acceptable excipient is selected from sorbitol and xylitol.

6. A solid oral composition comprising silodosin having a particle size distribution d₉₀ of 1 μm to 25 μm, more preferably from 10 μ to 20 μ.

7. The solid oral composition of claim 1, further comprising
sorbitol as a diluent, and
pregalantinized starch or croscarmellose sodium as the pharmaceutically acceptable excipient, wherein the solid oral composition is in the form of a capsule.

8. The composition according to claim 7, comprisinges 1 to 8 wt % of silodosin, 60 to 90 wt % of sorbitol and 0.1 to 5 wt % of sodium stearyl fumarate based on total weight of the composition.

9. The solid oral composition according to claim 1, wherein particle size distribution d₉₀ of silodosin is 1 μm to 25 μm

10. The composition according to claim 1, comprising either powder blend or granules prepared by wet granulation.

11. The composition according to claim 10, in the form of capsules or compressed tablets.

12. A method of making a silodosin composition comprising:
(i) sifting and blending silodosin with one or more pharmaceutically acceptable excipients to form a uniform blend, (ii) lubricating the uniform blend of step (i) with sodium stearyl fumarate to provide a lubricated blend, and (iii) filling the lubricated blend of step (ii) into capsules.

13. The composition according to claim 1, wherein the composition is free of sodium lauryl sulphate.

14. The composition according to claim 1, wherein the composition is free of magnesium stearate, calcium stearate, talc or a combination thereof.

15. A method of treating the signs and symptoms of benign prostatic hyperplasia in a patient in need thereof, comprising administering to a patient the composition of claim 1.

16. The solid oral composition according to claim 7, wherein particle size distribution d₉₀of silodosinis 1 μm to 25 μm.

17. Thecomposition according to claim 7, comprise either powder blend or granules prepared by wet granulation.

18. The composition according to claim 17, wherein said powder blend or granules were filled into capsules or compressed into tablets.

19. The composition according to claim 9, wherein the composition is free of sodium lauryl sulphate.

20. The composition according to claim 9, wherein the composition is free of magnesium stearate, calcium stearate, talc or a combination thereof.
